(19) United States
(12) Patent Application Publication
(10) Pub. No.: US 2026/0126393 A1
(43) Pub. Date: May 7, 2026

(54) METHOD FOR DETERMINING THE LITHIUM CONTENT IN A BIOLOGICAL FLUID

(71) Applicants: CENTRE NATIONAL DE LA RECHERCHE SCIENTIFIQUE, Paris (FR); UNIVERSITE DE FRANCHE-COMTE, Besancon (FR); CENTRE HOSPITALIER REGIONAL UNIVERSITAIRE DE BESANCON, Besancon (FR); INSTITUT NATIONAL DE LA SANTE ET DE LA RECHERCHE MEDICALE, Paris (FR)

(72) Inventors: Bruno WACOGNE, Traitiefontaine (FR); Charles-Louis AZZOPARDI, Besancon (FR); Bernard GAUTHIER-MANUEL, Besancon (FR); Karine CHARRIERE, Liesle (FR)

(21) Appl. No.: 19/117,444

(22) PCT Filed: Oct. 2, 2023
(86) PCT No.: PCT/EP2023/077268

§ 371 Date: Apr. 1, 2025

(30) Foreign Application Priority Data
Oct. 4, 2022 (FR) FR2210130

(51) Int. Cl. G01N 21/78; G01N 21/82; G01N 33/487

(52) U.S. Cl. CPC G01N 21/78; G01N 21/82; G01N 33/487; G01N 2201/0221

(57) ABSTRACT

Method for determining an amount of lithium contained in a biological fluid, said method comprising the steps consisting in adding, to a biological fluid sample of known volume, a given volume of a discriminating solution, the effect of which is to obtain a biological fluid solution buffered at a pH of between 6 and 8 and to bring about precipitation of at least a portion of the cations contained in the biological fluid sample except for the lithium ions. The method further comprises the steps consisting in depositing a given volume of biological fluid solution on an active layer (2) of an optode (1), said active layer comprising a chemical transducer of which at least one optical property is modified in the presence of lithium ions in the biological fluid solution, in measuring at least one characteristic of at least one light wave emitted or reflected by the active layer of the optode and in determining, from the at least one measured characteristic and from calibration data, an amount of lithium contained in the biological fluid.

### TECHNICAL FIELD

The present invention relates to the field of determining the amount of lithium contained in biological or body fluids by means of an optode.

More particularly, the present invention relates to the monitoring of lithium levels at home by persons who suffer from mood disorders and who receive a lithium treatment such as, but not limited to, bipolar disorder or in combination with other treatments for resistant depressive disorders.

### PRIOR ART

The product sold by FISIC under the trade name “Medimate Multireader” is known in the prior art. This device can be used by a user for measuring their level of lithium in the body. The measurement is accurate because same is based on electrophoresis. However, the analysis requires the collection of a blood sample from the user, which is an invasive procedure.

The use of an optode for the determination of the level of lithium in biological fluids is also known in the prior art. The document of Albero et al., “Novel flow-through bulk optode for spectrophotometric determination of lithium in pharmaceuticals and saliva”, Sensors and Actuators B, 145, (2010) 133-138 describes the determination of the concentration of lithium in saliva by means of an optode using spectrophotometry.

A further goal of the invention is to propose a method for determining the level of lithium in a biological fluid:
making it possible to determine the level of lithium at a concentration of less than 5 mM, and/or
making it possible to determine the level of lithium in the saliva, and/or
at low cost, and/or
simple to implement, and/or
allowing the user to carry out the process themselves and at home.

### PRESENTATION OF THE INVENTION

To this end, a method for determining a quantity of lithium contained in a biological fluid is proposed. The method comprises the steps consisting of:
obtaining, from a sample of biological fluid of known volume, a solution of biological fluid buffered at a pH comprised between 6 and 8, denoted as step A,
adding, to the solution of biological fluid, a given volume of a discriminating solution having the effect of leading to the precipitation of at least a part of the cations contained in the solution of biological fluid with the exception of lithium ions, denoted as step B,
depositing a volume, preferably a given volume, of solution of biological fluid on an active layer of an optode; said active layer comprises a chemical transducer of which at least one chemical property is modified in the presence of lithium ions in the solution of biological fluid, denoted as step C,
measuring at least one characteristic of one light wave emitted or reflected by the active layer of the optode, preferably by the chemical transducer,
determining, from at least one characteristic measured and from calibration data, a quantity of lithium contained in the biological fluid.

The solution of biological fluid may be any liquid or viscous solution, preferably aqueous, containing lithium ions.

Steps A, B can be implemented in any chronological order. Preferably, step C is implemented subsequently to steps A and B.

Preferably, steps A and B form a one and same step consisting in adding, to a sample of biological fluid of known volume, a given volume of a discriminating solution having the effect of:
obtaining a solution of biological fluid buffered at a pH comprised between 6 and 8, and
precipitating at least part of the cations contained in the sample of biological fluid with the exception of lithium ions.

Preferably, the discriminating solution has the effect of precipitating the di-cations contained in the solution of biological fluid, more particularly magnesium and calcium ions. Preferably, the discriminating solution precipitates the mono-cations contained in the solution of biological fluid, with the exception of lithium ions, more particularly potassium and sodium ions. Preferably, the discriminating solution precipitates all cations, with the exception of lithium ions, contained in the solution of biological fluid.

Preferably, obtaining a solution of biological fluid buffered at a pH comprised between 6 and 8 has the effect of dispensing with the influence of at least part of the cations contained in the solution of biological fluid, preferably the di-cations, preferably the calcium and magnesium ions, and the mono-cations, with the exception of lithium ions, contained in the solution of biological fluid, preferably potassium and sodium ions, on the uptake of lithium by the active layer of the optode.

Preferably, the biological fluid may be blood or saliva. In the context of monitoring the level of lithium at home by the user themselves, saliva is preferably the preferred biological fluid.

Preferably, the method does not comprise any invasive step of collecting the sample of biological fluid. The manufacturing method comprises a step of obtaining a biological fluid.

The term “given” can be defined as determined or known.

Preferably, the at least one optical property is modified compared to the amount of lithium in the solution of biological fluid. Preferably, the at least one optical property is modified either proportionally or not proportionally, but bijectively to the amount of lithium in the solution of biological fluid.

Optical property or characteristic of at least one light wave may refer to light intensity, hue, absorption or luminescence.

At least one characteristic of the at least one light wave may refer to at least one variation of at least one characteristic of the at least one light wave.

The optical property of the transducer may be an emission and/or a variation of an emission and/or a stopping of an emission of at least one light wave by the chemical transducer.

The characteristic of the at least one light wave emitted or reflected by the active layer can be defined as the optical property of the active layer.

Calibration data can be stored data. The calibration data can be a calibration curve or function, or a value or correspondence table.

Preferably, the step of obtaining the buffered solution of biological fluid is carried out by adding a volume, preferably a given volume, of a buffer solution not comprising cations, with the exception of hydronium ions, preferably not comprising di-cations, more particularly magnesium and calcium, else more preferably not comprising mono-cations, more particularly sodium and potassium ions, in the sample of biological fluid.

Preferably, the step of measuring at least one characteristic of at least one light wave emitted or reflected by the active layer of the optode is carried out by means of an optical measuring device. The optical measurement, using the optical measurement device, can be carried out by transmission or by reflection.

The step of adding a given volume of discriminating solution to the solution of biological fluid may cause a desolvation of at least a portion of the cations contained in the solution of biological fluid with the exception of lithium ions.

The method may comprise, prior to the step of adding the discriminating solution, a step of filtering the solution of biological fluid or the biological fluid on a porous membrane having a cut-off threshold of less than or equal to 50 μm.

Preferably, the discriminating solution is a buffer solution containing no cations with the exception of hydronium ions.

Preferably, the discriminating solution is a buffer solution not comprising dications.

Preferably, the discriminating solution is a buffer solution that does not comprise mono-cations with the exception of hydronium ions.

Preferably, the discriminating solution is a buffer solution that does not comprise magnesium and calcium ions.

Preferably, the discriminating solution is a buffer solution that does not comprise sodium and potassium ions.

Preferably, the buffer solution is a pH 7 buffer solution of tris(hydroxymethyl)aminomethane:HCl.

Preferably, the tris(hydroxymethyl)aminomethane:HCl molar ratio is 1:1.

The buffer solution may be a buffer solution of or comprising or containing tricine or N-(2-hydroxy-1.1-bis(hydroxymethyl)ethyl)glycine, bicin or [bis(2-hydroxyethyl)amino]acetic acid, HEPES or 4-(2-hydroxyethyl)-1-piperazine ethanesulfonic acid or MES or 2-(N-morpholino)ethanosulfonic acid.

Preferably, the discriminating solution is an aqueous solution comprising oxalate ions at a concentration greater than or equal to 1 milliMolar (mM), or 1 mmol.l⁻¹, preferably 5 mM.

The concentration of oxalate ions in the discriminating solution may be comprised between 1 mM and the solubility limit of oxalic acid in water. Preferably, the concentration of oxalate ions in the discriminating solution is 5 mM.

Preferably, the at least one characteristic of the at least one light wave emitted or reflected by the active layer of the optode comprises, preferably is, a hue value of the active layer.

The hue of the solution may be the Hue value in a colorimetric space or system, e.g. HSL or HSV.

Preferably, the method comprises a step of processing the at least one measured characteristic of the at least one light wave emitted or reflected by the active layer of the optode wherein the at least one measured characteristic comprises a set of colorimetric values in a colorimetric space, e.g. CIE XYZ, or another space, e.g. HSV, obtained from, or which is a function of, the CIE XYZ colorimetric space.

The set of colorimetric values can be, or can be represented as, a chromaticity diagram.

Preferably, the at least one measured characteristic comprises three colorimetric values. The set of colorimetric values may comprise the hue and/or the saturation and/or the luminance.

Preferably, the step of processing the at least one measured characteristic comprises:
a change of coordinate frame, within the colorimetric space, carried out on the two XY dimensions, then
a projection on the Y axis of the colorimetric space of the measured colorimetric values.

The change of coordinate frame may be a step of rotation, in the CIE XYZ colorimetric space, preferably reduced to two dimensions, denoted XY, by an angle comprised between 35° and 60° of the measured colorimetric values. The change of coordinate frame can be a principal component analysis or any other transformation.

Preferably, the step of processing the at least one measured characteristic is performed on two colorimetric values of the set of colorimetric values.

The change of coordinate frame, within the colorimetric space, can be performed on all three dimensions of the XYZ colorimetric space.

According to the invention, an optode is also proposed, preferably for the determination of the amount of lithium contained in a biological fluid, comprising:
a white support made of chemically inert material,
an active layer resting on the support, said active layer comprising a chemical transducer arranged so that at least one optical property is modified in the presence of a specific ionic species, preferably lithium ions,
a black layer made of chemically inert material comprising at least one opening forming a well arranged to receive a solution to be analyzed and intended to be in contact with the active layer.

Chemically inert may refer to a material that does not interfere or interfere little with the biological fluid or the medium containing the biological fluid and/or does not deteriorate or deteriorate little the optode, more particularly the active layer of the optode.

Inert may refer to chemically biocompatible. Biocompatible may refer to a material that does not interfere with and/or degrade the biological medium or the medium comprising the biological fluid with which same is intended to be brought into contact.

Resting on the support may refer to immobilized, hooked, integral or attached onto the support.

Specific may refer to determined or particular.

Preferably, the chemically inert material is a polymer.

Preferably, the black layer of chemically inert material rests, at least in part, preferably completely, on the active layer.

Preferably, the active layer of the optode comprises an ionophore and a chromophore.

The active layer may further comprise an additive, e.g. a lipophilic anionic additive.

Preferably, the chemically inert material is poly(methyl methacrylate) (PMMA).

The chemically inert material may be Teflon.

The chemically inert material may be inorganic. The chemically inert material may be a ceramic.

Preferably, the optode according to the invention is suitable, else preferably is particularly suitable, more preferably is designed and particularly advantageously is specially designed, for implementing the method for determining a quantity of lithium contained in a biological fluid according to the invention.

Any characteristic of the optode according to the invention can be directly transposed to the determination method according to the invention and vice versa.

According to the invention, it is also proposed to use an aqueous solution comprising oxalate ions at a concentration greater than 1 mM, preferably greater than 5 mM, and having a pH greater than or equal to 6 for the determination of the quantity of lithium contained in a biological fluid.

The concentration of oxalate ions in the aqueous solution may be between 1 mM and the solubility limit of oxalic acid in water. Preferably, the concentration of oxalate ions in the aqueous solution is 1 mM.

“Aqueous containing oxalate ions at a concentration greater than 1 mM and having a pH greater than or equal to 6 for the determination of the amount of lithium contained in a biological fluid” may refer to the use of said aqueous solution for the preparation of a solution of biological fluid intended to be analyzed for determining the amount of lithium contained therein.

### BRIEF DESCRIPTION OF FIGURES

Other advantages and particularities of the invention will become apparent upon reading the detailed description of examples used and of an embodiment which are in no way limiting, and of the following enclosed drawings:

FIG. 1a is a schematic representation of an embodiment of a portable optical measuring device coupled to the optode according to the invention in a configuration suitable for measuring the optical properties of the optode,

FIG. 1b is a schematic representation of the portable optical measuring device uncoupled from the optode,

FIG. 2 is a schematic representation of an optode illustrating the variation in hue of the active layer of the optode as a function of the concentration of lithium,

FIG. 3a is a representation of the xy components of the CIE xyY colorimetric space obtained from multispectral data measured on an optode according to the invention,

FIG. 3b is an enlargement of the xy components of the CIE xyY colorimetric space shown in FIG. 3a,

FIG. 4 is a representation of the data obtained by processing the XY components of the CIE XYZ colorimetric space shown in FIGS. 3a and 3b,

FIG. 5 is a representation of an optode comprising a single well according to the invention.

### DETAILED DESCRIPTION OF EMBODIMENTS

Since the embodiments described hereinafter are in no way limiting, variants of the invention may in particular be considered, comprising only a selection of characteristics described, isolated from the other characteristics described (even if such selection is isolated within a sentence comprising the other characteristics), if the selection of characteristics is sufficient to confer a technical advantage or to differentiate the invention with respect to the prior art. Such selection comprises at least one functional preference feature without structural details, or with only a part of the structural details if said part alone is sufficient to impart a technical advantage or to differentiate the invention from the prior art.

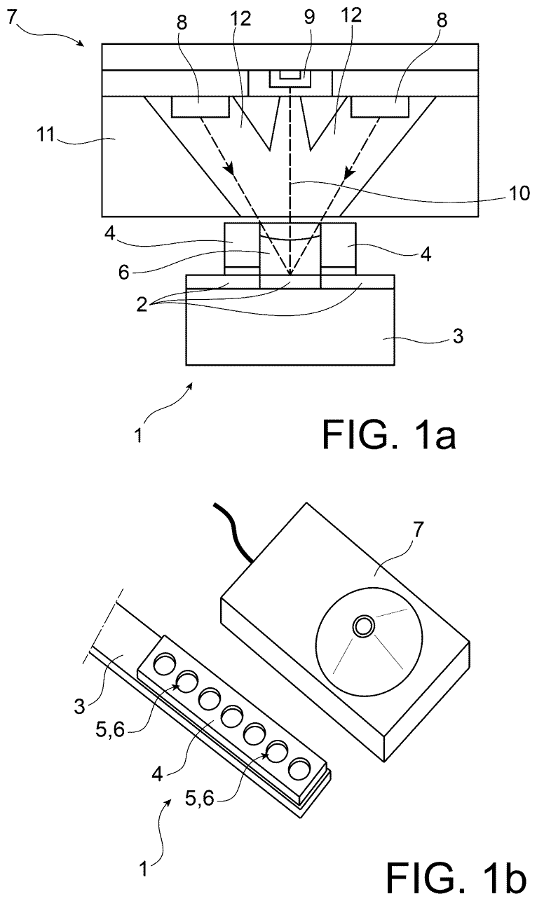

With reference to FIGS. 1 to 4, the method for determining the quantity of lithium contained in a liquid is presented. The embodiment relates to the determination of the amount of lithium in biological fluids and, more particularly, but not limited to, in saliva. Saliva is a fluid that does not require a delicate or invasive sampling step and can thus be collected at home by the user themselves. Mood stabilizing agents are among the different molecules available for treating bipolar disorders. Clinically, the main actions that qualify a molecule as a mood stabilizer are the effects thereof at both ends of the mood spectrum (depression and mania) and the ability thereof to maintain euthymia by preventing future mood instability. According to such factors, lithium is the best mood stabilizing agent and hence the reference agent. The advantage of the method according to the invention is that same is designed so that a user can implement same at home without the intervention of a third party and that the user can measure their level of lithium when is necessary by obtaining results in a short time. However, the method can also be implemented by a third party.

The saliva is preferably mechanically filtered through a membrane filter and membranes with cut-off thresholds of 0.45 μm and 0.8 μm were used. A volume of 1 ml of filtered saliva was used according to the non-limiting embodiment. The use of a membrane filter is not limiting and other filtration means could also be used.

Sodium, potassium, magnesium and calcium cations are present in most biological fluids. The inventors observed that the variability of the concentration of cations from one sample of biological fluid to another modifies the operating point of the optode used for the optical measurement and hence makes the determination of the concentration of lithium quite unreliable. The use of a chelating agent, EDTA (ethylenediaminetetraacetic acid), to complex the cations proved to be inconclusive. Thus, to overcome such problem, the method comprises the step consisting in obtaining a solution of biological fluid by adding a given volume of a discriminating solution to the sample of biological fluid, herein filtered saliva, of known volume. According to the embodiment, 1 ml of discriminating solution is added to 1 ml of filtered saliva. Saliva may contain residues and the filtration step aims to remove such residues in order to improve the reproducibility and reliability of the subsequent measurement performed with the optode 1. In practice, a volume comprised between 100 and 500 μl of saliva will be preferred.

According to the embodiment, the discriminating solution is a Tris/HCl buffer solution at pH 7 containing sodium oxalate at a concentration of 50 mM. Ammonium oxalate is preferred in that same does not add additional sodium ions.

The use of the discriminating solution is used for the production of a solution of biological fluid buffered at a pH comprised between 6 and 8. In such way, the exchanges between the lithium and the reactive surface of the optode are maximized, which makes it possible to optimize the colorimetric variation of the active layer 2 and thus the sensitivity of detection of lithium. Furthermore, the use of the discriminating solution also has the effect of removing mono-cations, more particularly calcium and magnesium ions, from the solution of biological fluid and thereby making reliable the determination of the level of lithium. Preferably, the mixture is conveyed via fluid channels or tubing from the filtration zone to the well(s) 6 of the optode 1.

The use of the discriminating solution also has the surprising effect of leading to a precipitation of at least part of the cations contained in the sample of biological fluid with the exception of lithium ions. More particularly, the use of the discriminating solution makes it possible to efficiently precipitate the di-cations contained in the sample of biological fluid, more particularly magnesium and calcium ions, and thereby make the determination of the level of lithium even more reliable.

After mixing the discriminating solution and the sample of biological fluid, 30 to 40 μl of the solution of biological fluid obtained are then deposited in a circular well 6 of an optode 1. The bottom of the well 6 is formed by the active layer 2 of the optode 1. The solution is left in contact with the active layer 2 between two and five minutes before the optical measurement is carried out. The active layer 2 of an optode 1 comprises a chemical transducer, at least one optical property of which is modified in the presence of lithium ions in the solution of biological fluid.

The measurement of at least one characteristic of at least one light wave coming from, i.e. emitted or reflected, by the active layer 2 of the optode 1 is then carried out, and the determination of the quantity of lithium contained in the biological fluid is then carried out on the basis of the at least one characteristic measured and on the basis of calibration data of the optode 1. Depending on the non-limiting embodiment, a calibration curve may form the calibration data.

According to the embodiment, the concentration of lithium in the sample is determined by colorimetric analysis of the active layer 2 of the optode 1. The measurement of the characteristic of the light waves coming from the active layer 2 of the optode 1 is carried out by a portable optical measurement device 7 illustrated in FIGS. 1a and 1b. The optical measuring device 7 is arranged to cooperate with the optode 1. The optical measuring device 7 is intended to be coupled reversibly with the well 6 of an optode 1. The optical measurement device 7 comprises a light source 8 and a multispectral sensor 9 of model AS7341 sold by the company AMS®, which measures the light reflected 10 by the optode 1. A processing unit, either connected or not connected to the measuring device, is arranged and/or configured to process the measured data. The measuring device 7 comprises two light-emitting diodes 8 of the model YJ-VTC-5730-G01-65 sold by the company YUJILEDS®, which are arranged to illuminate the active layer 2 of the optode 1 with a white light D65 CRI 98. The light beam 10 reflected by the active layer 2 is measured by the multispectral sensor 9. Preferably, the optical measurement device 7 comprises a cover 11 intended to be brought into contact with the black layer 4 of chemically inert material of the optode 1 which forms the outer or upper layer of the optode 1. The multispectral sensor 9 is placed opposite the optode 1 when the latter is coupled to the optical measurement device 7. The light coming from the LEDs 8 is “guided” at 45° in channels 12 provided in the cover 11 for illuminating an individual well 6 of the optode 1.

The data from the multispectral measurement are then converted by the processing unit into a three-dimensional colorimetric space and the value of concentration of lithium is determined. The step of converting the measured characteristics of the light wave coming from the active layer 2, or from the multispectral values, is a step well known to a person skilled in the art.

The measurement of at least one characteristic of at least one light wave coming from the active layer 2 of the optode 1 corresponds to the multispectral measurement.

The measurements or results presented hereinafter in the description were obtained by implementing the method described hereinabove and, in particular, by using the discriminating solution according to the invention.

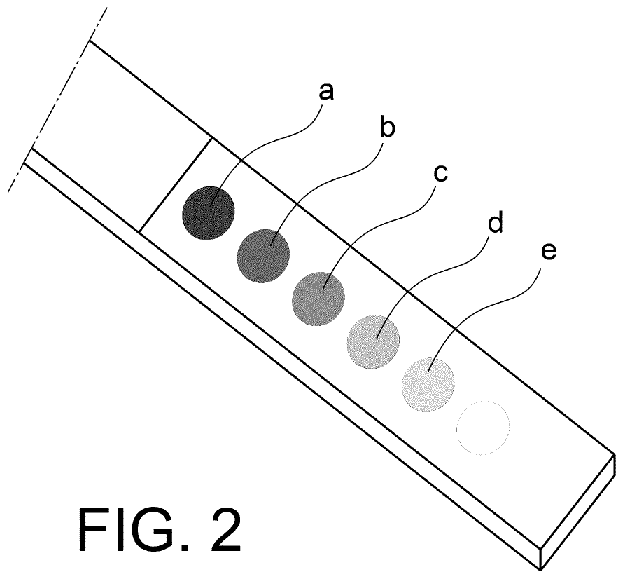

With reference to FIG. 2, the variations in hue of the active layer 2 of the optode 1 as acquired by photography of the optode 1 and as observable with the naked eye for concentrations of lithium of 0 (a), 1 (b), 2 (c), 3 (d) and 4 (e) mM in deionized water are illustrated. The level of lithium influences the hue of the active layer 2 of the optode 1. The results are obtained from an optode 1 not comprising a well 6. A drop of each solution of lithium of different concentration was deposited on the active layer 2. The optode 1 is preferably intended for experimental studies or for obtaining calibration data. The optode 1 intended to be used by a user will preferably comprise a single well 6 forming a consumable intended to be replaced. Such results show that it is possible to detect, with the naked eye, variations in hue of the active layer 2. Of course, it is also possible to detect very small variations in hue of the active layer 2, which are undetectable by the naked eye, by digitally analyzing the hue values converted from the spectral measurements made.

However, the variation in hue of the active layer 2 of the optode 1 in the therapeutic window is small and is difficult to perceive by the naked eye. In addition, other factors can modify the hue, such as the variation in thickness of the active layer 2 of the optode 1, the photobleaching of the chromophore of the active layer 2 and the aging of the optode 1. Variations in hue, even slight, disturb and distort the measurement. The inventors observed that the other components of the colorimetric space also vary with such perturbations. Thereby, the invention also consists in exploiting the information contained in all the components of the XYZ colorimetric space, in order to compensate for the measurement error.

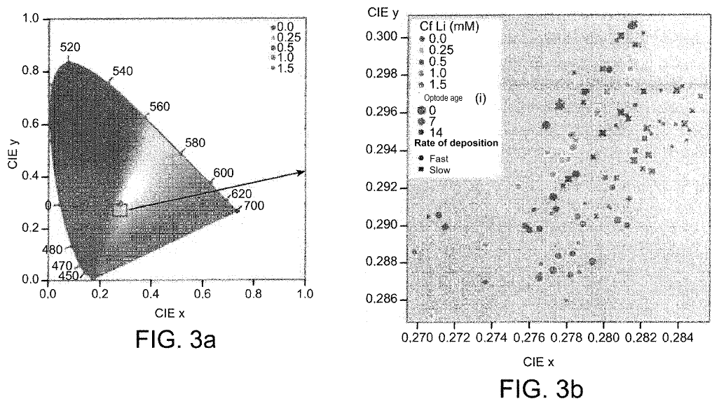

Thus, another embodiment providing greater detection sensitivity and better reproducibility of measurements, is presented. The chromaticity diagram, indicating the hue, corresponds to the xy components of the CIE xyY colorimetric space. The chromaticity diagram is a two-dimensional representation of the CIE XYZ colorimetric space without information on luminance. Measurements were made on the optode 1 from aqueous solutions of deionized water comprising 0.25, 0.5, 1 and 1.5 mM of lithium. In order to evaluate the stability of the light emission by the active layer 2 of the optode 1, the measurements were carried out on the same optode 1 on the first day of the manufacture thereof and on the seventh and fourteenth days after the manufacture thereof. Furthermore, the measurements were carried out on a plurality of distinct optodes 1 the active layers 2 of which were deposited at two distinct rates of deposition. A first rate of deposition of the active layer 2 by dip-coating is 50 mm/s, called the fast rate of deposition, and a second rate of deposition of the active layer 2 by dip-coating is 10 mm/s. The multispectral measurements performed in each case were converted into the CIE XYZ colorimetric space in three dimensions. With reference to FIGS. 3a and 3b, the chromaticity diagram, corresponding to the colorimetric values of the two xy dimensions of the CIE xyY colorimetric space, is shown. The chromaticity diagram provides information on the hue of the active layer 2.

In the CIE xyY colorimetric space, it is possible to apply a linear regression on the different measurements of lithium despite the variations of parasitic hues. However, since the regressions are not parallel, it is not possible to calculate a reliable color-dependent metric corresponding to the concentration of lithium.

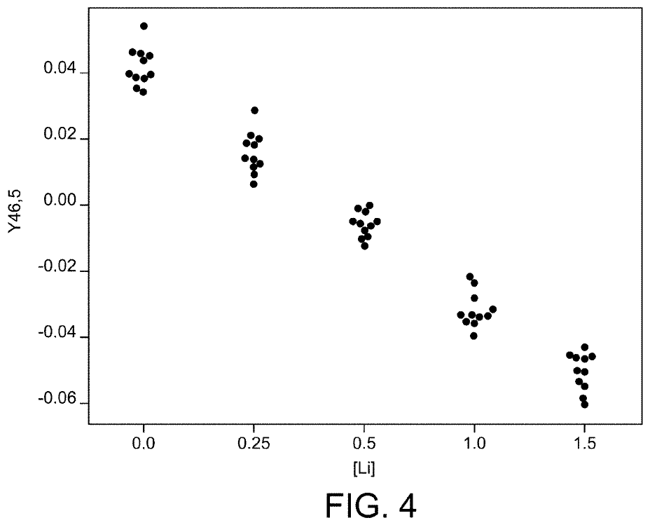

Also, a change of coordinate frame is first performed, within the CIE XYZ colorimetric space, on the two XY dimensions. The change of coordinate frame can be e.g. a principal component analysis or a rotation of the components in the CIE XYZ colorimetric space. Depending on the embodiment, a rotation between 45 and 47° is performed within the CIE XYZ space. Such range of values is not limiting and will be adapted according to the experimental conditions used, such as e.g. the composition of the discriminating solution, more particularly the concentration of salts in the buffer solution, the type of buffer, the pH of the solution or the type of counterion accompanying the oxalate, the biological fluid targeted or the composition of the active layer 2 used. The angle of rotation will be mainly comprised between 35° and 60°. Following the change of coordinate frame, a projection of the CIE XYZ colorimetric space of the XY colorimetric components is made onto the Y axis. The result of the projection is shown FIG. 4. Following the change of coordinate frame and the projection, it can be observed that the colorimetric values projected on the Y axis are a function of and vary linearly with respect to the concentration of lithium on the x-axis. The data thereby obtained can be used as calibration data during the implementation of the method for determining the level of lithium in a biological fluid.

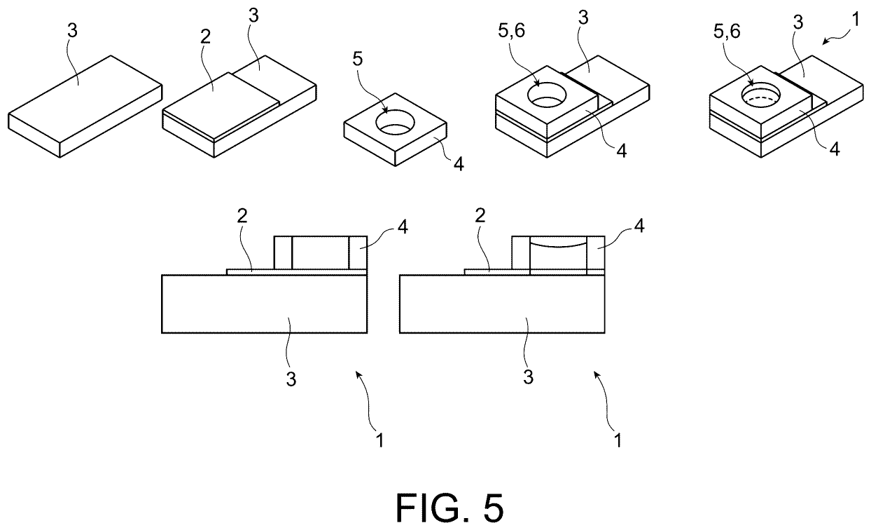

With reference to FIG. 5, a preferred embodiment of the optode 1 according to the invention intended to be used by a user is illustrated. The optode 1 described hereinbelow is the optode that was used in the implementation of the method described hereinabove. The optode 1 comprises a white support 3 made of chemically inert material. According to the embodiment, the material used is a polymer, more particularly PMMA. It has been observed that PMMA significantly lengthens the lifetime of the active layer 2 and decreases the variations in light emission of the active layer 2 over time. The optode 1 can be used for at least fourteen days without any consequent chromophore variations being observed. As comparative examples, it has been observed that the use of polyethylene as support 3 imparts a lifetime or use of one day on the active layer 2 and that the use of poly(vinyl chloride) as support 3 makes the optode 1 unusable because same is not functional. However, it is also possible to envisage using other polymers such as e.g. Teflon, polyetheretherketone or polyurethane or other materials such as e.g. ceramics.

According to the non-limiting embodiment, the active layer 2 resting on the support 3 comprises poly(vinyl chloride), DOS (bis(2-ethylhexyl)sebacate), Lithium ionophore VIII, Chromoionophore 1 (ETH 5294) and Potassium tetrakis(4-chlorophenyl)borate (K-TCPB). In practice, the active layer 2 is deposited by dip-coating on the support 3 from a solution obtained by dissolving in 25 ml of tetrahydrofuran, 417 mg of PVC, 911 μl of DOS (bis(2-ethylhexyl)sebacate), 23.7 mg of Lithium ionophore VIII, 11.1 mg of Chromoionophore 1 (ETH 5294) and 12.6 mg of Potassium tetrakis(4-chlorophenyl)borate (K-TCPB). The thickness of the active layer 2 obtained after dip-coating is comprised e.g. between 1 and 3 μm. In practice and in a non-limiting manner, the white PMMA support 3, in the form of a tongue according to the embodiment, is dipped in the solution at a rate of 10 mm/s, then left for 0.5 s in the solution, then is raised at a rate of 10 mm/s. The support can be dried for about 30 min so that the solvent, THF according to the embodiment, evaporates.

According to the non-limiting embodiment, the optode 1 also comprises a black layer 4 of chemically inert material, PMMA according to the embodiment, comprising at least one opening forming a well arranged to receive the solution to be analyzed intended to be in contact with the active layer 2. As an example, a circular well 6 may have a diameter of 4.5 mm and a height, from the active layer 2 to the top of the well 6, of 3 mm.

The black layer 4 of chemically inert material comprises a through opening 5. The black PMMA layer 4 rests on the active layer 2. The black color of the chemically inert material comprising the through opening 5 makes it possible to limit the influence of light reflections and of the ambient light on the multispectral measurement.

Of course, the invention is not limited to the examples which have just been described and many modifications can be brought to said examples without departing from the scope of the invention.

Thereby, in variants of the embodiments described above that can be combined with one another, the aqueous solution, and/or the use thereof for determining the quantity of lithium contained in a biological fluid, comprises oxalate ions at a concentration greater than 5 mM and has a pH greater than or equal to 6.

In addition, the different characteristics, forms, variants and embodiments of the invention may be associated with one another in various combinations insofar as same are not incompatible or exclude each other.

1. A method for determining an amount of lithium contained in a biological fluid, comprising:
adding, into a sample of biological fluid of known volume, a given volume of a discriminating solution having the effect of:
obtaining a solution of biological fluid buffered at a pH comprised between 6 and 8, and
thereby causing a precipitation of at least a part of the mono- and di-cations contained in the sample of biological fluid with the exception of lithium ions;
depositing a given volume of solution of biological fluid on an active layer of an optode, wherein said active layer comprises a chemical transducer of which at least one optical property is modified in the presence of lithium ions in the solution of biological fluid;
measuring at least one characteristic of at least one light wave emitted or reflected by the active layer of the optode; and
determining, from at least one characteristic measured and from calibration data, a quantity of lithium contained in the biological fluid.

2. The method according to claim 1, comprising, prior to the step of adding the discriminating solution, a step of filtering the solution of biological fluid on a porous membrane having a cut-off threshold of less than or equal to 50 μm.

3. The method according to claim 1, wherein the discriminating solution is a buffer solution not comprising magnesium and calcium and/or sodium and potassium cations.

4. The method according to claim 1, wherein the discriminating solution is a buffer solution comprising no di-cations and/or mono-cations except for hydronium ions.

5. The method according to claim 1, wherein the buffer solution is a pH 7 buffer solution of tris(hydroxymethyl)aminomethane:HCl.

6. The method according to claim 1, wherein the discriminating solution is an aqueous solution comprising oxalate ions at a concentration greater than 1 mM.

7. The method according to claim 1, wherein the at least one characteristic of the at least one light wave emitted or reflected by the active layer of the optode comprises a hue value of the active layer.

8. The method according to claim 1, further comprising a step of processing the at least one measured characteristic of the at least one light wave emitted or reflected by the active layer of the optode:
wherein the at least one characteristic measured comprises a set of colorimetric values in a colorimetric space,
comprising:
a change of coordinate frame, within the colorimetric space, carried out on the two dimensions of the colorimetric space, denoted by XY, then
a projection on the Y axis of the colorimetric space of the measured colorimetric values.

9. An optode for implementing the method according to claim 1, wherein said optode comprises:
a white support made of chemically inert material;
an active layer resting on the support, said active layer comprising a chemical transducer arranged so that at least one optical property is modified in the presence of a specific ionic species; and
a black layer made of chemically inert material comprising at least one opening forming a well arranged to receive a solution to be analyzed and intended to be in contact with the active layer.

10. The optode according to claim 9, wherein:
the active layer comprises an ionophore and a chromophore, and
the chemically inert material is poly(methyl methacrylate) (PMMA).
